(19) United States
(12) Patent Application Publication
(10) Pub. No.: US 2009/0105577 A1
(43) Pub. Date: Apr. 23, 2009

(54) Device For Detecting Electrical Potentials Using Frontal Electrodes

(76) Inventors: Jianping Wu, Mississauga (CA); Ronald Leon Kurtz, Oakville (CA)

Correspondence Address: BERESKIN AND PARR, 40 KING STREET WEST, BOX 401, TORONTO, ON, M5H 3Y2, CA

(21) Appl. No.: 12/272,051

(22) Filed: Nov. 17, 2008

Related U.S. Application Data
(63) Continuation of application No. PCT/CA2007/000873, filed on May 17, 2007.
Application No. PCT/CA2007/000873 is a continuation-in-part of application No. 11/435,938, filed on May 18, 2006.

(30) Foreign Application Priority Data
May 18, 2006 (CA) 2547371

(51) Int. Cl. A61B 5/0478

(52) U.S. Cl. 600/383; 600/544

(57) ABSTRACT

Embodiments relate to devices for detecting electrical potentials corresponding to biological signals. The device comprises a flexible member for positioning over a forehead area of a human head and first, second and third electrodes located on the flexible member. The first electrode is located in a projecting portion of the flexible member to be positioned adjacent a nasion area of the head. The second electrode is to be positioned over a first lateral forehead area. The third electrode is to be positioned over a second lateral forehead area opposite the first lateral forehead area.

### TECHNICAL FIELD

Embodiments relate to a device for detecting electrical potentials using frontal electrodes. In particular, embodiments involve positioning of at least three electrodes on human forehead positions.

### BACKGROUND

For medical diagnostic purposes, it can be useful to determine the sleep stages experienced by a person during sleep. Such sleep stage determination has traditionally been performed in a laboratory setting, in which the patient is asked to sleep while undergoing the testing. Under such conditions, the patient is likely to experience abnormal sleep patterns.

The sleep stage determination is performed by affixing a plurality of electrodes on the patient's scalp at various standard positions according to the 10-20 system of electrode placement. Some electrodes are positioned to sense electroencephalographic (EEG) signals, while other electrodes may be positioned to detect electromyographic (EMG) signals or electrooculographic (EOG) signals. The EEG, EMG and EOG signals may be provided to a processing system, including, for example, a neural network for use in determining the stage of sleep experienced by the person according to the detected signals.

Embodiments attempt to address or ameliorate one or more of the disadvantages or shortcomings associated with existing devices for use in sleep stage determination, or to at least provide a useful alternative thereto.

### SUMMARY

Embodiments relate to devices for detecting electrical potentials corresponding to biological signals. The device comprises a flexible member for positioning over a forehead area of a human head and first, second and third electrodes located on the flexible member. The first electrode is located in a projecting portion of the flexible member to be positioned adjacent a nasion area of the head. The second electrode is to be positioned over a first lateral forehead area. The third electrode is to be positioned over a second lateral forehead area opposite the first lateral forehead area. The second and third electrodes have a separation of between 35 mm and 200 mm.

Conductors may be formed on the flexible member for electrically coupling the first, second and third electrodes to an output connector. The second and third electrodes may be located on opposite lateral wings of the flexible member and the output connector may be coupled to one of the lateral wings. The first electrode may be positioned immediately above the nasion, between the eyebrows.

The device may further comprise a fourth electrode located on the flexible member intermediate the second and third electrodes for positioning over a central forehead area. The fourth electrode may be positioned above the first electrode and both the first and fourth electrodes may be aligned with or positioned along a vertical centerline of the head. The fourth electrode may be positioned lower on the forehead than an Fz electrode position. The fourth electrode may act as a reference or ground electrode. If the fourth electrode acts as a ground electrode, the first electrode may act as a reference electrode.

The second and third electrodes may be positioned on the forehead higher than and laterally beyond Fp1 and Fp2 positions. The second, third and fourth electrodes may be positioned along a line extending laterally across the forehead. The first, second and third electrodes may be positioned in a triangular configuration, such as an isosceles triangular configuration. The first, second, third and fourth electrodes may be positioned in a T-shaped configuration.

The conductors may comprise a printed flexible material. The conductors may comprise silver and/or silver chloride, for example. The conductors may be formed on a substrate of the flexible member. The conductors and substrate may be covered by an insulation material in areas where the electrodes are not formed on the flexible member. The insulation material may have a double-sided adhesive foam layer thereon for adhering the device to the forehead and fixing the electrodes in position against the forehead.

Each electrode may have a layer of conductive gel disposed thereon for facilitating conduction of electrical signals or potentials on or below the skin to the respective electrode. The device may have a protective layer on an underside thereof for protecting the conductor gel and adhesive foam prior to application of the device to the forehead. The protective layer is readily removable from the device.

The second and third electrodes may have a lateral separation of about 70 to 110 mm. The separation may be 80 to 100 mm. The separation may be about 90 mm.

The first and fourth electrodes may have a separation of about 35 to 55 mm. The separation may be about 40 to 50 mm. The separation may be about 44 mm.

The second and third electrode may have a portion of adhesive material disposed around the respective electrode for affixing the respective electrode to the skin of the forehead.

The biological signals may be generated during a sleep study. The device may be used to determine a sleep stage of a wearer of the device. The device may be used to determine an ischemia of a wearer of the device. The device may be used to determine a level of consciousness of a wearer of the device.

A method of detecting electrical potentials corresponding to biological signals may be comprised of positioning the device over the forehead area so that the first electrode is positioned adjacent the nasion area, the second electrode is positioned over the first lateral forehead area and the third electrode is positioned over the second lateral forehead area; and detecting the electrical potentials using the first, second and third electrodes.

Another particular embodiment relates to a sensing device for use in sleep stage determination using frontal electrodes. The device comprises: a flexible member for positioning over a forehead area of a human head, the flexible member having opposed lateral wings and a projecting portion; a plurality of electrodes located on the flexible member for positioning laterally across the forehead; and an electrode located on the projecting portion for positioning adjacent a nasion area.

Yet another particular embodiment relates to a sensing device for use in sleep stage determination using frontal electrodes. The device comprises: a flexible member for positioning over at least part of the forehead of a human head, the flexible member being approximately T-shaped; and a plurality of electrodes located on the flexible member for positioning over areas on or adjacent the forehead, at least one electrode being located on the flexible member at a bottom of the T-shape for positioning adjacent a nasion area when the device is positioned on the head in an upright position.

### BRIEF DESCRIPTION OF THE DRAWINGS

Embodiments are described in further detail below by way of example only, with reference to the accompanying drawings, in which:

FIG. 1 is a front view of a sensing unit shown located on a human forehead, according to some embodiments;

FIG. 2 is an illustrative side cross-section of the sensing unit of FIG. 1, taken along the line A-A;

FIG. 3A is a representative side view of a human head, showing standard electrode positions and electrode positions according to some embodiments;

FIG. 3B is a representative plan view of a human head corresponding to FIG. 3A;

FIG. 4 is a schematic representation of the relative positions of electrodes on the sensing unit; and

FIG. 5 is an illustrative partial side cross-section of the sensing unit according to further embodiments.

### DETAILED DESCRIPTION

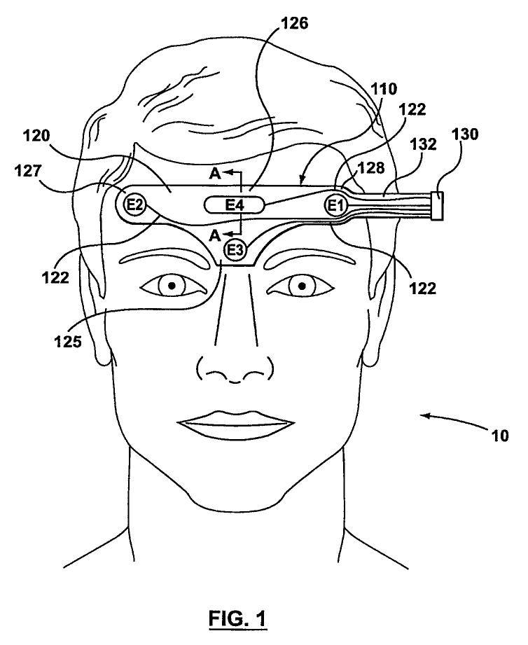

Referring now to FIGS. 1 to 5, embodiments of a device for use in measuring biological signals are shown and described. In the drawings and description, like reference numerals are used to indicate like features, functions and/or elements as between the drawings.

In this description, reference to terms implying a directional orientation, such as lateral, vertical, below, above or downward, are intended to be viewed as if the embodiments are positioned on a forehead of a human head, while that head is upright. Accordingly, “vertical” is intended to denote directions from the top of the skull toward the neck, while “lateral” is intended to denote positions or directions to one side of a midline of the head extending along the frontal line of symmetry of the face (i.e. perpendicular to vertical). Thus, “lateral” as applied to the forehead means extending across the forehead between the eyebrows and the hairline and, depending on the shape of the particular forehead, extending around toward the upper temple area.

It should be understood that terms used herein that imply direction or orientation, such as those mentioned above, are used for ease of description only and are not intended to be a limitation on the described embodiments when they are not in use on the forehead.

Referring in particular to FIG. 1, there is shown a sensing unit 110 positioned on the forehead of a human head 10. Sensing unit 110 has an electrode array including four electrodes E1, E2, E3 and E4 formed thereon for overlying exposed skin surfaces of the forehead and nasion areas. Electrodes E1, E2, E3 and E4 are used to detect EEG, EMG and EOG signals, for example during a sleep study or for determining the occurrence of our ischemia or determining the level of consciousness of the wearer.

Sensing unit 110 comprises a flexible plate-like member 120 formed roughly in a squat T-shape when viewed from the front while worn on the head 10. A lower portion 125 of flexible member 120 projects downwardly from the substantially laterally extending body of flexible member 120. Lower portion 125 houses electrode E3 so as to be positioned to at least partly overlie the nasion area or an area adjacent thereto. Depending on the forehead structure of the head 10, electrode E3 may be positioned slightly above the nasion area, but generally on a centre line extending vertically through the forehead intermediate the eyes and eyebrows.

Electrodes E1, E4 and E2 are spaced laterally across sensing unit 110. Electrode E4 acts as a ground electrode relative to the measured signals from electrodes E1, E2 and E3. Electrodes E1 and E2 are positioned in laterally extending wings 127 and 128 located on respective right and left sides of the head 10 (as seen from the patient's perspective). Electrodes E1 and E2 and wings 128 and 127 are positioned widely (laterally) so that, for most forehead sizes and structures, the electrodes E1, E2 are positioned on the forehead above and laterally beyond a vertical centerline through the eyes. The greater lateral spacing of electrode E1 and E2 allows the sensing of a greater amount of relevant EEG data.

Ground electrode E4 is positioned generally centrally on sensing unit 110 within a central area 126 of flexible member 120 that connects to the internally extending wings 127 and 128 and lower portion 125.

As shown in FIG. 1, flexible member 120 has a connector limb 132 extending from a left side (seen from the patient's perspective) thereof and a connector 130 at an end of connector limb 132. Connector 130 is arranged to electrically couple conductors 122 extending through sensing unit 110 to a processing unit (not shown), thereby forming an electrical connection between the processing unit and the electrodes E1, E2, E3 and E4 to which conductors 122 are electrically coupled.

According to one embodiment, sensing unit 110 is formed mostly of flexible materials for placement on a forehead structure and for generally conforming to the shape of the forehead structure. Certain parts of sensing unit 110 (for example, those around the electrodes) have an adhesive substance, such as a foam adhesive layer, on an underside thereof, for affixing the sensing unit 110 to the forehead prior to conducting the sleep study. Flexible circuitry, in the form of conductors 122 extends through sensing unit 130 between the electrodes E1 to E4 and connector 130. Thus, sensing unit 110 can be used with forehead structures of varying shapes and sizes due to its flexibility and ability to conform and adhere to such varying forehead structures, as required.

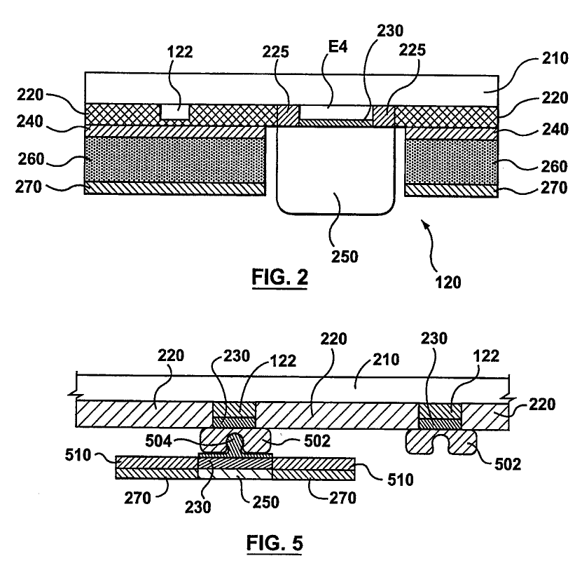

Sensing unit 110 is shown in FIG. 2 in partial cross-section, taken along line A-A of FIG. 1. Flexible member 120 employs a substrate 210 of a flexible material such as a medical grade polyester film (or other material having similar properties).

Flexible member 120 has a substrate 210, which forms the top (or upper or outer) layer facing away from the forehead. Substrate 210 has sufficient rigidity to form the base for flexible circuitry to be printed thereon and enable subsequent conductive and insulative layers to be formed thereon, while having sufficient flexibility to enable the entire flexible member 120 to bend to generally conform to the shape of the forehead to which it is to be affixed.

The substrate 210 may be about 3 to 8 thousandths of an inch thick, for example. Adhesive 270 is used to affix at least a part of the flexible member 120 to the forehead structure. This adhesive 270 is provided on a layer of medical grade adhesive foam 260 of about 1/32 of an inch thickness. The foam 260 is adhered to an insulation layer 220 on the substrate 210 on one side with a relatively strong adhesive 240 and has adhesive 270, which is of relatively less strength, on the opposite side for removable attachment to the test subject. The electrodes E1 to E4 may comprise a silver or silver chloride layer formed on the substrate. The substrate 210 has flexible circuit tracings formed thereon for constituting the conductors 122 between electrodes E1 to E4 and output connector 130. Such circuit tracings may comprise silver and preferably have a dielectric layer (such as insulation layer 220) formed thereover.

Prior to affixation to the forehead, sensing unit 110 may have backing sheets on those parts of sensing unit 110 that have an adhesive substance on their undersides for adhesion to the skin. Each such backing sheet is removed immediately prior to adhesion of the relevant part of sensing unit 110 to the corresponding forehead areas. For electrodes E1 to E4, an area of conductive gel, such as hydrogel, is interposed between the respective electrode and the skin surface (instead of the adhesive foam), for facilitating conductivity of electrical signals between the electrodes E1 to E4 and the skin.

Sensing unit 110 is a generally flat device, as viewed from the user's perspective, prior to affixation to the test subject. However, sensing unit 110 does have several layers, as described above. In use of sensing unit 110, and with the backing sheets removed, the adhesive foam parts and electrodes E1 to E4 are positioned to lie against the skin. These skin contact surfaces may be conveniently referred to as being formed on the underside of the sensing unit 110. Printed labeling, including affixation instructions, may be provided on the side of sensing unit 110 that does not contact the skin.

Electrodes E1 to E4 are formed on substrate 210, either directly or on a thin priming or separation layer (not shown) coating the underside of substrate 210. Electrodes E1 to E4 are electrically coupled to output connector 130 via conductors 122 in the form of flexible circuit tracings formed on substrate 210. As with electrodes E1 to E4, conductors 122 may be directly formed on substrate 210 or may be separated therefrom by a priming or separation layer. Portions of flexible member 120 that are not to be exposed to the forehead (such as conductors 122) are covered by insulation layer 220.

In the embodiment shown in FIG. 2, electrode E4 comprises a silver chloride layer 230 on its outer face for facilitating conductivity with the skin via a conductive gel in contact with electrode E4. The conductive gel is provided as a liquid hydrogel and is impregnated into a porous foam sponge 250 that contacts the skin when the sensing unit 110 is positioned on the patient's forehead. Sponge 250 is adhered to substrate 210 by an adhesive layer 225 disposed around the electrodes. In order to allow for compression of the sponge during skin contact, a gap is formed on either side of the sponge 250 between the sponge 250 and the foam layer 260.

In an alternative embodiment, a substantially more viscous conductive gel can be used instead of the sponge 250 and liquid hydrogel, in which case the adhesive layer 225 and the compression gap are not required. The above impregnated sponge arrangement and the viscous hydrogel arrangement are both commercially available from Vermed, Inc. of Bellows Falls, Vt., USA.

Adhesive layer 270 and conductive sponge 250 are covered by a protective backing sheet or layer (not shown) so that the adhesive and conductive qualities of the adhesive layer 270 and conductive sponge 250 are preserved until application of flexible member 120 to the forehead. The total thickness of substrate 120 may be in the range of 0.7 to 1.5 millimeters, approximately.

The embodiments shown in FIGS. 2 and 5 are not to scale, is for purposes of illustration only and some variations or modifications may be made, depending on the specific requirements of the sensing unit embodiment and methods of forming it.

While the sensing unit embodiments shown and described herein generally show a unitary flexible member including two wings and projecting portion, each of the areas or portions of the sensing unit having electrodes may be formed on a separate, but connected, substrate.

In an alternative embodiment of sensing unit 110, metallic disk electrodes may be used with a flexible member formed of molded plastic, such as a polyvinylchloride (PVC) plastic. In such an embodiment, the plastic is preferably relatively thin and flexible to accommodate the contours of the wearer's forehead, while having sufficient structural integrity and rigidity to maintain the electrodes in their respective positions. Such a molded plastic flexible member may employ a suitable adhesive to secure it in place on the forehead. Alternatively, or in addition, a strap or other mechanical means may be used to secure the sensing unit 110 in place on the wearer's forehead.

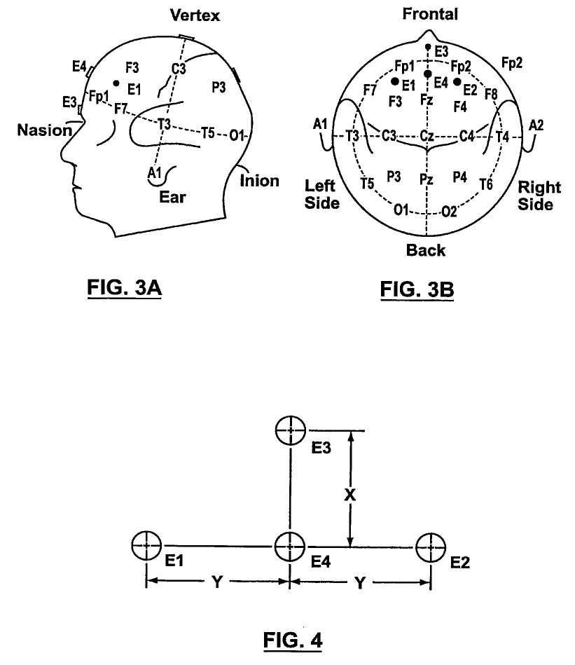

Referring in particular to FIGS. 3A, 3B and 4, the positioning of electrodes E1, E2, E3 and E4 is described in further detail. FIGS. 3A and 3B indicate the likely positions of electrodes E1 to E4 on a human head, relative to the standard 10-20 electrode positions. As can be seen from FIGS. 3A and 3B, reference electrode E3 is positioned adjacent the nasion area. Electrode E3 is located on flexible member 120 so that, for most forehead structures, it will be positioned immediately above the nasion and in between the eyebrows. Electrode E3 is thus positioned on the vertical centerline of the head in a position lower than the line extending through frontal positions Fp1 and Fp2.

Electrode E4 is positioned on the midline (vertical centerline) below frontal position Fz but above the frontal line extending through frontal positions Fp1 and Fp2. Electrodes E3 and E4 are separated by a distance X, as shown in FIG. 4, where X may be about 35 to 70 mm. In some embodiments X may be between about 35 mm and 55 mm. In some embodiments, X may be about 40 to 50 mm. In further embodiments, X may be about 44 mm. The distance X can be measured as the separation of electrodes E3 and E4 or it can be measured as the vertical separation of electrode E3 from the lateral line extending between electrodes E1 and E2. This latter configuration may be applicable where electrode E4 is not used or is not positioned along the lateral line between electrodes E1 and E2.

As shown in FIG. 4, electrodes E1 to E4 are arranged in a T-shaped configuration, with reference electrode E3 at a bottom of the T and electrodes E1, E2 and E4 forming the top line of the T. In alternative embodiments, the electrode configuration need not be strictly T-shaped. For example, ground electrode E4 may be shifted up or down so that it is not strictly in line with electrodes E1 and E2.

Further, electrodes E1, E2 and E3 are arranged in a triangular configuration, where the distance between electrodes E1 and E3 is the same as the distance between electrodes E2 and E3, but may or may not be the same as the distance between electrodes E1 and E2. Thus, electrodes E1, E2 and E3 are arranged in an isosceles triangular configuration. This configuration allows the electrodes to be arranged in sensing pairs E1-E3 and E2-E3 to sense EEG, EOG and EMG potentials, while sensing electrode pair E1-E2 is also arranged to sense EEG, and EOG potentials. The E1-E3 and E2-E3 electrode pair orientations may be configured to be substantially orthogonal to each other.

Electrodes E1 and E2 are each laterally separated from electrode E4 by a distance Y that may be the same as distance X or may be different therefrom. The total distance (2Y) between electrodes E1 and E2 is, according to some embodiments between about 35 and 200 mm. In other embodiments, the separation of electrodes E1 and E2 is between about 50 and 150 mm. In further embodiments, the separation is between about 70 and 110 mm. In other embodiments, the separation of electrodes E1 and E2 is about 80 to 100 mm. In further embodiments, the separation is about 90 mm.

In determining possible configurations and separations of electrodes E1, E2, E3 and E4, there is a lower bound of about 35 mm separation between any two electrodes. This lower bound is determined experimentally as the minimum separation needed to produce two distinct signals. It is also constrained physically by the dimensions of the electrodes and the need to keep them sufficiently electrically separated to avoid creation of a salt bridge through the skin.

An upper bound of about 200 mm for the lateral spacing of electrodes E1 and E2 is also applicable and takes into account the desirability of avoiding the hairline and of avoiding electrode placement on the side of the skull. It is generally desirable to place an electrode in such a way that it does not make contact with a pillow when the wearer is sleeping on his or her side. Otherwise, contact of the electrode with the pillow will introduce artifacts in the sensed electrical potentials at the electrode when the patient moves (known as a motion artifact) and may result in an uncomfortable pressure point for the wearer.

An upper bound on the vertical separation of the third and fourth electrodes may be about 70 millimeters, which is on average the maximum distance between the nasion and the hairline at the lateral centre of the forehead.

Between the upper and lower bounds of the separations of the electrodes are preferred dimensions for distance Y and distance X as described above, although some variation of these distances can be used while remaining within the upper and lower bounds described above. Such preferred distances may be, for example, about 90 millimeters for the separation between electrodes E1 and E2, which equates to 2Y. This spacing provides sufficient spacing of electrodes E1 and E2 so that, on average, those electrodes are positioned laterally beyond the vertical centre lines of the eyes and the fp1, fp2 positions, while not extending so far as to cause electrodes E1 and E2 to be placed on the sides of the head 10. For a lateral separation of about 90 millimeters between electrodes E1 and E2, a vertical separation of about 44 or 45 millimeters between E3 and the lateral line extending between electrodes E1 and E2 may be used, regardless of whether reference electrode E4 is employed. Such a vertical separation of electrode E3 from electrodes E1 and E2 allows electrodes E1, E2 and E3 to be configured roughly as an isosceles right angled triangle, where the line between electrodes E1 and E3 is roughly perpendicular to the line between electrode E2 and E3. Such a configuration is not essential, however, and variations of the triangular configuration of electrodes E1, E2 and E3 may be employed to similar effect.

Electrodes E1 and E2 are located on flexible member 120 so as to be positioned on the forehead at forehead locations above and laterally beyond standard frontal positions Fp1 and Fp2, respectively. This wider spacing of electrodes E1 and E2 across the frontal area allows for a greater range and quality of EEG signal activity to be detected than if the standard Fp1 and Fp2 positions were used. This greater range compensates for the lack of the commonly used reference electrode positioned at A1 or A2 behind the ear.

The specific configuration of electrodes E1, E2 and E3 allows for simultaneous sensing of EEG, EOG and EMG signals using a single electrode assembly on a flexible member that is easily applied by a patient to his or her own forehead prior to self-initiation of the sleep study. Thus, sensing device 110 is easily applied in a home setting without the need for the patient to be studied in an artificial environment and without the need for a medical technician to affix the electrodes to the patient's head 10.

In alternative embodiments of sensing unit 110, one or more of electrodes E1 to E4 may comprise a needle electrode specifically configured for EMG signal detection. Alternatively, or in addition, one or more of electrodes E1 to E4 may have a wireless transmitter associated therewith (instead of a conductor 122) for transmitting wireless signals to a nearby receiver, such as is described in co-pending, co-owned U.S. patent application Ser. No. 11/130,221, entitled “Wireless Physiological Monitoring System”, filed May 17, 2005, the entire contents of which is hereby incorporated by reference.

Although not shown in FIG. 1, some embodiments of sensing unit 110 may have a strap attachable to each lateral wing 127, 128 for securing sensing unit 110 to head 10. Such a strap may be in addition or alternative to adhesive 270 for securing sensing unit 110 in place. In place of a strap, other means for securing the sensing unit to the head 10 may be employed.

In further embodiments, as depicted in FIG. 5, electrodes E1 to E4 are removably attachable to flexible member 120. In these embodiments, electrodes E1 to E4 are formed as disk-shaped electrodes that have male snap connector parts 504 on a back surface thereof for engaging a corresponding female snap connector part 502 positioned on flexible member 120. In these embodiments, conductors 122 are electrically coupled (for example by a silver chloride layer 230) to the female snap connector parts 502, which form a mechanical and electrical connection with the electrodes via the male snap connector parts 504 on each electrode.

In these embodiments, the underside of flexible member 120 may or may not employ an adhesive to affix the flexible member 120 to the forehead. If no adhesive is used, a strap or band can be used to secure the flexible member 120 in the appropriate location. In order to affix the electrodes E1 to E4 to the appropriate locations on the forehead and nasion areas, each electrode is provided with an annular portion of adhesive foam or other substance around the outside of the conductive contact surface (conductive gel 250) of the electrode. Alternatively, the conductive gel on the contact surface of the electrodes may have sufficient adhesive properties to obviate the use of adhesive foam portions around the electrodes. Examples of commercially available “snap connector” electrodes that can be used with these electrodes include those sold by Vermed under their Versa-Trode™ product line.

The removably attachable electrode embodiments allow the flexible member 120 to be reusable while the electrodes can be disposed of after each use. In these embodiments, the flexible member 120 may be comprised of a material having greater flexibility and/or deformation properties than the polyester film or PVC described above. A suitable material may comprise a cloth or other woven material. Alternatively, the flexible member 120 may be comprised of a relatively more rigid material, such as PVC, although this rigidity is not strictly required if each electrode is held in place on the skin by the portion of adhesive material 270 surrounding it.

FIG. 5 illustrates embodiments of sensing unit 110 that employ “snap on” electrodes for electrodes E1, E2, E3 and optionally E4. Each such snap on electrode comprises the male connector part 504 connected to a substrate 510 that has adhesive layer 270 on its outward skin facing surface. The male connector part 504 may have a silver chloride layer 230 deposited on its outward (skin facing) side, over which is positioned a conductive gel or foam layer 250. Thus, the male connector part 504, silver chloride layer 230 and female connector part 502 effectively form the electrode, with the male connector part 504 being carried on substrate 510 for removable connection to sensing unit 110 via female connector part 502.

Further, according to some embodiments, in order for sensing unit 110 to accommodate foreheads of different sizes, substrate 210 may have a group of closely spaced or adjacent female connector parts 502 (in electrical connection with a single conductor 122), one of which will receive the male connector part 504 of the electrode when the sensing unit 110 is in use.

Although FIG. 5 depicts one form of snap-fitting connection, it should be understood that other forms of electromechanical connection may be employed to provide removable connection of the electrode to the sensing unit 110.

While the configuration of the electrode array of sensing unit 110 is arranged in a T-shaped configuration, alternative configurations, for example where the central ground electrode E4 is positioned higher or lower, may be employed. However, electrode configurations that necessitate placement of one of the electrodes over a hair-covered part of the scalp are less desirable than those that allow placement of the electrodes over hairless areas of the scalp. Thus, shapes analogous to a T-shape, such as a cross-shape, Y-shape or other shapes having laterally extending wings and a downwardly projecting portion, may be employed to a similar effect to the embodiments using a T-shaped electrode configuration and a flexible member. In one embodiment, the lateral wings of the flexible member 120 may extend further laterally and droop down to cover the temple areas on either side of the head. This allows additional electrodes to be placed over the temple areas for increased EEG sensing capability.

While sensing unit 110 is described as being applicable for use in sleep stage determination, it should be understood that it can also be used in connection with other apparatus or software to record other results of diagnostic significance. One example of such other apparatus is a mask for providing continuous positive airway pressure (CPAP) to the patient, such as is described in U.S. patent application Ser. No. 11/131,284. Embodiments may also be used within the context of an intensive care unit (ICU), for example to assist in detection of a seizure, stroke, ischemia, burst-suppression or brain hemorrhage or for use in determining a level of consciousness, sedation or delirium of a patient.

According to alternative embodiments, additional sensors, which may be electrodes or other forms of sensors, may be provided for positioning at other locations on the head. For example, an additional electrode may be placed behind or in front of the ear or ears, for use as active or reference electrodes. Such additional sensors may be coupled to flexible member 120 for electrical connection to the processing unit via connector 130. Alternatively, a separate connector may be used for electrically coupling the additional sensor or sensors to the processing unit.

While certain embodiments described herein contemplate the use of four electrodes E1 to E4 located on the flexible member 120, it should be understood that, for each of those four electrodes, more than one electrode may be used in place of the single electrode. In still further embodiments, the sensing unit 110 may employ more than four electrodes at various positions on the flexible member 120. In a further alternative embodiment, the ground electrode E4 may be omitted or its position varied.

1. A device for detecting electrical potentials corresponding to biological signals, comprising:
a flexible member for positioning over a forehead area of a human head;
a first electrode located on a projecting portion of the flexible member for positioning adjacent a nasion area of the head;
a second electrode located on the flexible member for positioning over a first lateral forehead area; and
a third electrode located on the flexible member for positioning over a second lateral forehead area opposite the first lateral forehead area;
wherein the second and third electrodes have a separation of between 35 mm and 200 mm.

2. The device of claim 1, wherein the separation is between 50 and 150 mm.

3. The device of claim 2, wherein the separation is between 70 and 110 mm.

4. The device of claim 3, wherein the separation is between 80 and 100 mm.

5. The device of claim 4, wherein the separation is about 90 mm.

6. The device of any one of claims 1 to 5, wherein conductors are formed on the flexible member for electrically coupling the first, second and third electrodes to an output connector.

7. The device of claim 6, wherein the conductors comprise a printed flexible material.

8. The device of claim 6 or claim 7, wherein the second and third electrodes are located on opposed lateral wings of the flexible member.

9. The device of claim 8, wherein the output connector is coupled to one of the lateral wings.

10. The device of claim 9, wherein the flexible member comprises a flexible limb extending from the one lateral wing and having the output connector coupled to the conductors at an end of the limb.

11. The device of any one of claims 1 to 10, wherein the second and third electrodes are located on the flexible member for positioning higher on the forehead than Fp1 and Fp2 electrode positions.

12. The device of any one of claims 1 to 11, further comprising a fourth electrode located on the flexible member intermediate the second and third electrodes for positioning over a central forehead area.

13. The device of claim 12, wherein the fourth electrode is located on the flexible member for positioning above the first electrode.

14. The device of claim 13, wherein the first and fourth electrodes are located on the flexible member for positioning along a vertical centerline of the head.

15. The device of any one of claims 12 to 14, wherein the fourth electrode is located on the flexible member for positioning lower on the forehead than a Fz electrode position.

16. The device of any one of claims 12 to 15, wherein the second, third and fourth electrodes are positioned along a line.

17. The device of any one of claims 12 to 16, wherein the first, second, third and fourth electrodes are positioned in a T-shaped configuration.

18. The device of any one of claims 12 to 17, wherein the first and fourth electrodes have a separation of between 35 and 70 mm.

19. The device of claim 18, wherein the separation of the first and fourth electrodes is between 40 and 50 mm.

20. The device of claim 19, wherein the separation of the first and fourth electrodes is about 44 mm.

21. The device of any one of claims 1 to 20, wherein the second and third electrodes are located on the flexible member for positioning laterally beyond respective Fp1 and Fp2 electrode positions.

22. The device of any one of claims 1 to 21, wherein the first, second and third electrodes are positioned in an isosceles triangular configuration.

23. The device of any one of claims 1 to 22, wherein the first, second and third electrodes are removably connectable to the flexible member.

24. The device of claim 23, wherein each first, second and third electrode comprises a connection part for electrically and mechanically connecting to a corresponding part on the flexible member.

25. The device of claim 24, wherein each first, second and third electrode has a portion of adhesive material disposed around the respective electrode for affixing the respective electrode to the skin of the forehead.

26. The device of claim 24 or claim 25, wherein the flexible member comprises a plurality of said corresponding parts grouped together, wherein the connection part of the first, second or third electrode is connected to one of the plurality of corresponding parts.

27. The device of any one of claims 1 to 22, wherein each of the first, second and third electrodes is formed on a substrate of the flexible member.

28. The device of any one of claims 1 to 22, wherein the flexible member has an adhesive layer disposed on at least a part of an underside of the flexible member adjacent each of the first, second and third electrodes.

29. The device of any one of claims 1 to 28, wherein the first electrode is located on the flexible member for positioning immediately above the nasion and between the eyebrows.

30. The device of any one of claims 1 to 29, wherein the flexible member is approximately T-shaped.

31. The device of any one of claims 1 to 30, wherein a vertical separation of the first electrode and the second and third electrodes is between 35 and 70 mm.

32. The device of claim 31, wherein the vertical separation is between 35 and 55 mm.

33. The device of claim 32, wherein the vertical separation is between 40 and 50 mm.

34. The device of claim 33, wherein the vertical separation is about 44 mm.

35. The device of any one of claims 1 to 34, wherein the biological signals are generated during a sleep study.

36. Use of the device of any one of claims 1 to 35 to determine a sleep stage of a wearer of the device.

37. Use of the device of any one of claims 1 to 35 to determine an ischemia of a wearer of the device.

38. Use of the device of any one of claims 1 to 35 to determine a level of consciousness of a wearer of the device.

39. A method of detecting electrical potentials corresponding to biological signals, comprising:
positioning the device of any one of claims 1 to 35 over the forehead area so that the first electrode is positioned adjacent the nasion area, the second electrode is positioned over the first lateral forehead area and the third electrode is positioned over the second lateral forehead area; and
detecting the electrical potentials using the first, second and third electrodes.
